(19) United States
(12) Patent Application Publication
(10) Pub. No.: US 2021/0145318 A1
(43) Pub. Date: May 20, 2021

(54) SYSTEM FOR DETECTING A GAIT DISORDER OF A USER AND ASSOCIATED METHODS

(71) Applicants: ZHOR TECH, Nancy (FR); DIGITSOLE, Nancy (FR)

(72) Inventor: Karim OUMNIA, Nancy (FR)

(21) Appl. No.: 17/045,268

(22) PCT Filed: Apr. 8, 2019
(86) PCT No.: PCT/FR2019/050824

§ 371 Date: Oct. 5, 2020

(30) Foreign Application Priority Data
Apr. 6, 2018 (FR) FR1870414

(51) Int. Cl. A61B 5/11; A61B 5/00; A61B 5/103

(52) U.S. Cl. CPC A61B 5/112; A61B 5/4082; A61B 2562/0219; A61B 5/1038; A61B 2560/0214; A61B 5/6807

(57) ABSTRACT

The invention relates to a system 1 of characterizing the gait of a user to obtain a value representative of the evolution of the gait of said user, including a pair 10 of soles, the soles 11, 12, constituting said pair 10 of soles, each including an electronic box 100, 101, 102, each electronic box 100, 101, 102 comprising: an inertial platform 110, 111, 112, a data processing module 120, 121, 122, a data storage module 130, 131, 132, and a power source 160, 161, 162; said system including a data comparison module 140, 141, 142 configured to compare the at least one biomechanical parameter to reference biomechanical parameters and calculate a value representative of the evolution of the gait of the user; said data comparison module being carried by the electronic boxes or the external terminal; and each electronic box including a first communication means.

The invention relates to the field of shoes and more generally to that of footwear. The present invention relates more particularly to a system for detecting a gait disorder in a user. The invention also relates to a method of detecting a gait disorder in a user of a detection system.

### PRIOR ART

Shoes, and more specifically soles, whether inside or outside, essentially have the original role of protecting the foot from the ground. Their shape varies according to fashion and its vagaries to make room for a multitude of by-products and functions.

Shoes can be for relaxation, formal, sports, medical, professional, or simply recreational use. Thus, a shoe consists mainly of, on the one hand, a sole, the lower part which protects the sole of the feet, more or less raised at the back by the heel, and, on the other hand, the upper, the upper part which envelops the foot. It can be limited to the ankle or it can be a high shoe. The sole can be made in two parts. An upper sole layer in direct contact with the user's foot and a lower sole layer in direct contact with the ground or more generally the outside environment. A shoe may also include a removable insole. In this particular case, this sole also consists of at least one upper sole layer and one lower sole layer.

New technologies have been accompanying new needs and the world of footwear has been part of this movement. The development of electronics has led to the appearance of so-called connected soles and shoes, which have a wide range of functions. Very generally, these connected soles or shoes can be autonomous and contain a rechargeable battery. They can be connected to an external terminal by a wired system or wireless connection.

Among the functions offered by existing connected soles or shoes, we can mention the function of heating the foot to one or more given temperatures, determined by the manufacturer; we can also mention those, described in document US2017/0188950, equipped with pressure sensors and an accelerometer and which allow a smartphone connected via Bluetooth to deliver statistics on the physical activities of their wearer such as the number of steps taken.

There are also soles or shoes that can record biological information about their user. For example, document WO2017023868 relates to a device which, thanks to force or pressure sensors, provides information on the biomechanics of the user's walk. Similarly, document US2015/257679 presents a system of monitoring a runner's gait for measuring the forces exerted by a person's feet, in particular through force or pressure sensors, in order to train athletes to run better and to respect defined training parameters.

Nevertheless, these devices based on the presence of numerous sensors distributed in the sole (for example pressure sensors) have a shorter service life and often a relatively high thickness that can limit the use of these soles. In addition, calculations are generally not performed in real time. Thus, there is a need for a system that can quickly and reliably monitor a user's gait while being compact and ensuring high impact resistance.

Finally, these technical solutions do not allow for regular and efficient monitoring of the evolution of a user's locomotion, or even to identify anomalies likely to correspond to a risk of developing a malformation or pathology. In this sense, a system for the treatment of osteoarthritis has been proposed that allows modification of muscle activation patterns to treat knee pain associated with osteoarthritis (WO2017/023864). Nevertheless, data processing is done exclusively at a computing terminal and the soles are used for raw data acquisition. Moreover, this system does not allow the user's gait to be characterized in such a way as to be able to follow its evolution.

Thus, despite the variety of technological solutions offered, no user has access to soles or shoes that allow him/her to follow the evolution of his/her health condition from data collected directly from his/her feet. Indeed, as a general rule, the state of the art characterized by these various devices only allows the user to have information on his/her performance or on the characteristics of his/her immediate external environment.

Thus, there is a need for new systems to detect a user's gait disorder.

### Technical Problem

The invention aims to overcome the disadvantages of the prior art. In particular, the invention aims at providing a system for the detection of a user's gait disorder, said system being reliable, robust, and allowing the gait to be monitored in real time and over a longer period of time thanks in particular to an improved autonomy.

The invention, furthermore, aims at providing a method of detecting a gait disorder, where said method can be implemented in real time and essentially at the sole. Where this method allows to establish a value of at least one biomechanical parameter representative of the user's gait and to calculate an alphanumeric value representative of the evolution of the gait, in a quick, simple way, and not necessarily requiring the intervention of a specialist in the health field. It should be noted that this method is not intended to replace the general practitioner or specialist and does not make a diagnosis.

### BRIEF DESCRIPTION OF THE INVENTION

To this end, the invention relates to a system of characterizing the gait of a user to obtain a value representative of the evolution of the gait of said user, including a pair of soles and an external terminal, the soles constituting said pair of soles, each including an electronic box, each electronic box comprising:
an inertial platform configured to generate a set of data on the gait of a user of the pair of soles,
a data processing module configured to transform the generated data set into at least one biomechanical parameter,
a data storage module configured to store the at least one biomechanical parameter, and
a power source;
said system including a data comparison module configured to compare the at least one biomechanical parameter to reference biomechanical parameters and to calculate a value representative of the evolution of the gait of the user;
said data comparison module being carried by the electronic boxes or the external terminal; and
each electronic box comprising a first communication means configured so that the electronic box of at least one of the soles is adapted to transmit the at least one biomechanical parameter and/or the value representative of the evolution of the gait of the user to the external terminal.

Such a system allows to reliably monitor a user's gait. Indeed, the presence of a pair of soles, each including a box protecting an inertial platform, makes it possible to monitor the movement of each of the feet independently. The inertial platform will analyze, in at least three dimensions, the user's posture, movements, locomotion, balance, and environment, and more generally everything that will be qualified as his/her gait. The inertial platform will not only be able to note the different positions, but also to detect deficiencies or anomalies that appear in the user's locomotion, pattern, or more generally walk. In addition, the presence of the electronic box containing all the electronic components required for autonomous operation, such as all the sensors, including calculation modules and a power source, allows to increase the robustness of the system. This box can advantageously be unique, compact, and miniaturized.

Moreover, contrary to the systems proposed in the prior art, calculation is carried out here at the sole via a data processing module which can correspond to the firmware (“firmware” in Anglo-Saxon terminology) of an electronic board. In this way, the data is processed in real time at the electronic box, compared, and can then be transferred for visualization on an external terminal. Such a system allows to reduce the load on the memory of the storage module and can therefore increase the autonomy of the system.

Finally, the calculation of a value representative of the evolution of the user's gait allows, from a characterization of the user's gait, to give indications on his/her health condition, such as the effectiveness of a treatment, the appearance of a gait disorder, or a risk of the appearance of a gait disorder.

According to other optional features of the system:
said at least one biomechanical parameter is selected from: stability of the foot during the flight phase, step roll-forward, step length, step width, step angle, stride length, stride width, and step trajectory;
each electronic box further includes a second communication means configured so that the electronic box of a first sole is adapted to, preferably configured to, communicate with the electronic box of a second sole, and so that at least one of the data processing modules is configured to transform sets of data generated from the two soles constituting the pair of soles into at least one biomechanical parameter. Such a configuration allows to produce in real time more relevant biomechanical parameters for the study of the user's gait.
each electronic box also includes other sensors, which may be in particular a magnetometer, a barometer, a temperature sensor, and an altimeter. Preferably, each electronic box also includes other sensors, which may be in particular a magnetometer, a barometer, and an altimeter. The values generated by these additional sensors can be used to improve the accuracy of the value representing the evolution of the user's gait, or to provide additional information.
the transformation by the data processing module comprises the segmentation of the data into a plurality of step phases. Such segmentation allows for a better analysis of the user's gait. In earlier devices, it is not performed within the sole as is the case in the present invention.
the data processing module is adapted to, preferably configured to, calculate the values of at least two of the following biomechanical parameters: stability of the foot during the flight phase, step roll-forward, step length, step width, step angle, stride length, and/or stride width.
the reference biomechanical parameters to which the at least one biomechanical parameter is compared are biomechanical parameters previously generated by a same user of the system. This allows the user's gait to be monitored and a mobility disorder in the user to be detected early. Indeed, the gait is characterized over time and the data generated at different times are compared.
the data processing module is adapted to, preferably configured to, calculate an asymmetry between the biomechanical parameters of a right leg with respect to the biomechanical parameters of a left leg.
the reference biomechanical parameters to which the at least one biomechanical parameter is compared are predetermined biomechanical parameters, associated with one or more mobility disorders. Thus, it is possible to identify gait disorders by comparing the calculated parameters to reference data.
a second electronic box is configured to transmit, to a first electronic box, data generated by its inertial platform or one or more biomechanical parameters, and the first electronic box is then configured to generate a biomechanical parameter so-called synchronized in particular from:
   the least one biomechanical parameter obtained by the first electronic box, and
   data generated by the inertial platform of the second electronic box or one or more biomechanical parameters calculated by the second electronic box.
Thanks to these characteristics, it is possible to obtain a fine analysis of the user's gait and to identify disorders that may be inaccessible with biomechanical parameters generated from the data of a single box. This embodiment is particularly advantageous because it allows access to fine characterizations of the gait while using an energy-efficient on-board sensor system.
the data processing module or the comparison module is further configured to calculate a combinatorial pattern of biomechanical parameters. Such a characteristic allows to identify disorders that can be difficult to identify by conventional biomechanical parameters.
the data processing module is adapted to, preferably configured to, calculate the variability of the biomechanical parameters associated with one or both legs.
Such information can be of great interest in the context of quantifying or characterizing the user's gait.
the data processing module is adapted to, preferably configured to, establish a profile of biomechanical parameters of the user including at least one of the following parameters: step length, step angle, impact force, pace, and time of flight.
the data comparison module, of the electronic box or of the external terminal, preferably of the electronic box, is adapted to, preferably configured to, generate a data item selected from: an efficiency index of a care protocol, a data item of the nature of the support, a data item of the step profile, a data item of the walking technique, a data item of the support area, and a data item of a corrective solution.
the data comparison module, of the electronic box or of the external terminal, preferably of the electronic box, is configured to generate an efficiency index of a care protocol. Thus, a user and his/her doctor can identify in real time a deviation in the effectiveness of the treatment, which can be a sign of a lack of medication or the appearance of a new physiological state to be taken into account for the treatment.
the data comparison module, of the electronic box or of the external terminal, preferably of the electronic box, is configured to generate a data item of a corrective solution. For example, an orthopedist can thus use the generated data so as to design a suitable sole.
the data storage module is configured to store at least part of the transformed data but not to store the data generated by the inertial platform. Thus, the performance of the electronic boxes is not reduced by data to be stored.

The invention further relates to a method of characterizing the gait of a user implementing a quantification system including a pair of soles and an external terminal, the soles, constituting said pair of soles, each including an electronic box, each electronic box comprising an inertial platform, a data processing module, a data storage module, a first communication means, and a power source, said system including a data comparison module carried by the electronic boxes or the external terminal, said method including the following steps:
Generating, by the inertial platform, a set of data on the gait of a user of the pair of soles,
Transforming, by the data processing module, the generated data set into at least one biomechanical parameter,
Storing, by the data storage module, at least one biomechanical parameter,
Comparing, by the data comparison module, the at least one biomechanical parameter to reference biomechanical parameters,
Calculating, by the data comparison module, a value representative of the evolution of the gait of the user,
Transmitting, by a first communication means of at least one of the soles, the at least one biomechanical parameter and/or the value representative of the evolution of the gait of the individual to the external terminal.

The invention also relates to a method of designing orthopedic soles including the following steps:
a step of implementing a method of quantifying or characterizing the gait according to the invention, and
a step of defining the shape and ergonomics of orthopedic soles depending on the monitoring data obtained during the implementation stage.

Other advantages and features of the invention will appear upon reading the following description given by way of illustrative and non-limiting example, with reference to the appended figures which represent:

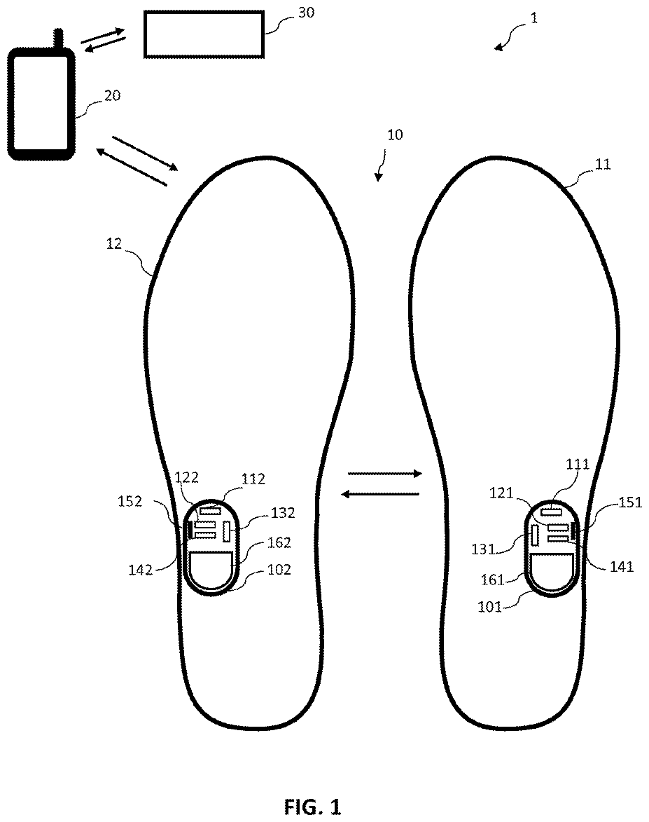

FIG. 1, a longitudinal section view as seen from above of two soles each containing a cavity which will give way to a box placed on the outer side of said sole, each of the antennas of the boxes being located on the outer side of each of the boxes, according to an embodiment of the invention.

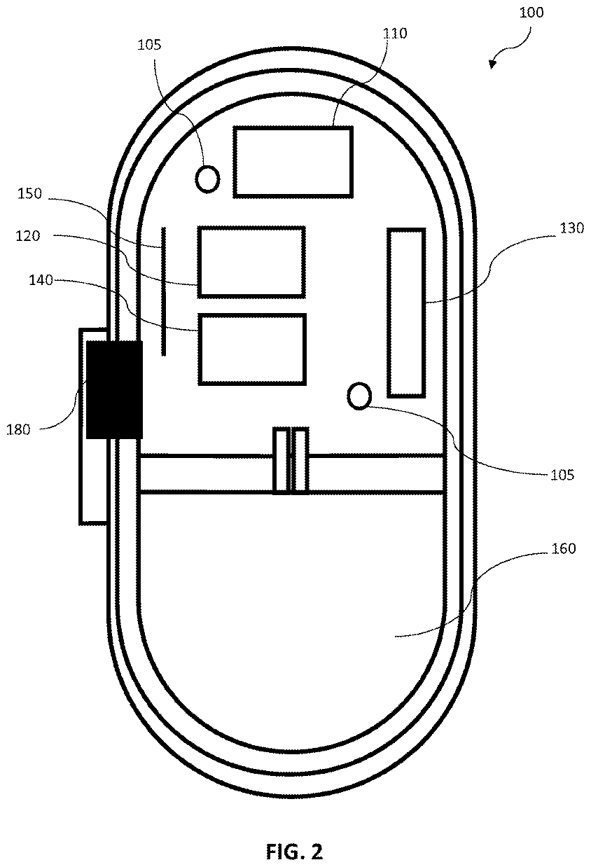

FIG. 2, an open electronic box as seen from above comprising in particular an electronic board, a rechargeable battery, a connector, and an antenna.

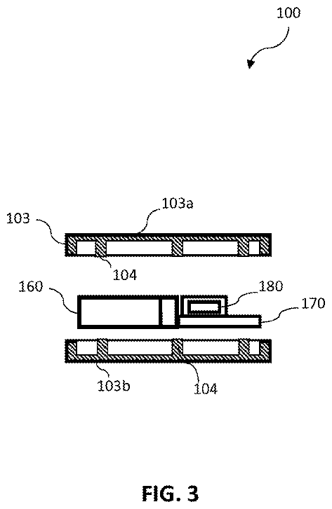

FIG. 3, an electronic box, in exploded and profile section view, comprising in particular a rechargeable battery, an electronic board, as well as a two-part outer casing.

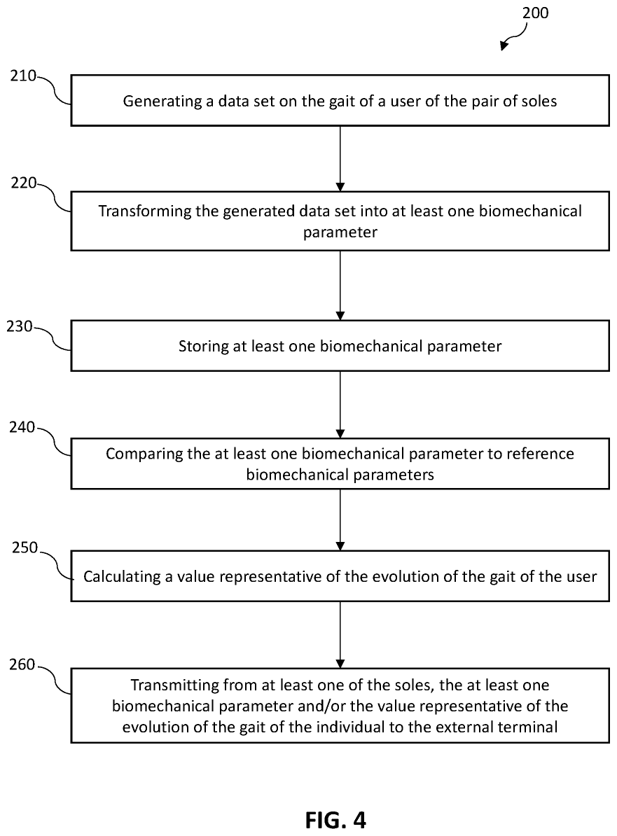

FIG. 4, a diagram of the method of characterizing the gait according to the invention.

### DESCRIPTION OF THE INVENTION

By “sole” is meant an object for separating the user's foot from the ground. A shoe may include an upper sole layer in direct contact with the user's foot and a lower sole layer in direct contact with the ground or more generally the outside environment. A shoe may also include a removable insole.

In the following description, “gait”, within the meaning of the invention, corresponds to the user's posture, movements, locomotion, and balance. The balance corresponds to the postural balance linked to the stability of the body and more particularly to the stability of the user's center of gravity.

The “gait quantification” corresponds, within the meaning of the invention, to the assignment of one or more values, for example a score, a ranking, or a mark to a trajectory or a movement of a user's foot. This gait quantification allows to obtain one or more biomechanical parameter values representative of the gait and can be performed based on many different linear or non-linear size scales (for example 1, 5, 10, 100). In this sense, the quantification of the gait within the meaning of the invention is equivalent to the characterization of the gait. In particular, the characterization of the gait includes quantifying, measuring, analyzing, and monitoring the evolution of the walking biomechanical parameters.

By “biomechanical parameter” and more particularly by “parameter calculated from movement data” is meant, within the meaning of the invention, the result of a transformation of the measured trajectory of a user's foot into one or more values.

By “reference biomechanical parameter” is meant a reference value, for example obtained from previous gait data from the user. The reference biomechanical parameter may also come from threshold values determined according to particular conditions and which may be associated with a particular gait or from values measured in persons, the gait of which has been previously qualified and the status of which is known. The status can for example relate to sports performance or pathological predispositions.

By “value representative of the evolution of the user's gait” is to be understood one or more values, for example a score, a ranking, or a mark, assigned to the user's gait in comparison with a reference. This reference can, for example, correspond to a value previously obtained for this user or a predefined threshold type value. This representative value can also be used to assign an individual to a group. The quantification according to the invention can be performed, in particular, by implementing a scoring algorithm generated from a learning method. The value representing the evolution of the user's gait can be a quantitative or qualitative value. For example, it can be a numeric value, a text value, or an alphanumeric value.

By “model” or “rule” or “algorithm” is to be understood, within the meaning of the invention, a finite sequence of operations or instructions for calculating a value through a classification or partitioning of the data within predefined groups Y and for assigning a score or ranking one or more data within a classification. The implementation of this finite sequence of operations allows, for example, to assign a label Y to an observation described by a set of characteristics or parameters X, using for example the implementation of a function f likely to reproduce Y, having observed X.

Y=f(X)+e

where e symbolizes noise or measurement error.

By “supervised learning method” is meant, within the meaning of the invention, a method for defining a function f from a base of n labelled observations (X_{1 . . . n}, Y_{1 . . . n}) where Y=f (X)+e.

By “unsupervised learning method” is meant a method aiming to prioritize data or divide a data set into different homogeneous groups, where the homogeneous groups share common characteristics without labeling the observations.

By “diagnosis” is meant the detection and/or identification in an individual of a disease, whatever the stage. In particular, the diagnosis allows to determine whether a subject has a neurological, neuromuscular, joint, muscular, or podiatric pathology.

Within the meaning of the present invention, by “prognosis” is meant the prediction of the evolution of a disease. In particular, “prognosis” refers to the assessment of the susceptibility to develop the disease, and/or the susceptibility to progression to a more advanced stage, and/or the risk of complications and exacerbations, and/or its outcome, etc.

“Assessment of disease progression” corresponds to the analysis over time of the evolution of the previously diagnosed or prognosticated pathology. Such monitoring overtime can allow the selection, validation, and/or adaptation of a treatment. It also allows to determine the intensity of clinical follow-up required for the patient. This follow-up can determine at any time whether treatment is necessary.

By “process”, “calculate”, “determine”, “display”, “transform”, “extract”, “compare” or more broadly “executable operation” is meant, within the meaning of the invention, an action performed by a device or processor unless the context indicates otherwise. In this respect, operations relate to actions and/or processes in a data processing system, for example a computer system or an electronic computing device, which manipulates and transforms data represented as physical (electronic) quantities in the memories of the computer system or other devices for storing, transmitting, or displaying the information. These operations can be based on applications or software.

The terms or expressions “application”, “software”, “program code”, and “executable code” mean any expression, code, or notation, in a set of instructions designed to cause data processing to perform a particular function directly or indirectly (for example after an operation of conversion to another code). Examples of program code may include, but are not limited to, a subprogram, function, executable application, source code, object code, library, and/or any other sequence of instructions designed for running on a computer system.

By “processor” is meant, within the meaning of the invention, at least one hardware circuit configured to perform operations according to instructions contained in a code. The hardware circuit can be an integrated circuit. Examples of a processor include, but are not limited to, a central processing unit, a graphics processor, an application-specific integrated circuit (ASIC), and a programmable logic circuit.

By “coupled” is meant, within the meaning of the invention, connected, directly or indirectly with one or more intermediate elements. Two members can be coupled mechanically, electrically, or linked by a communication channel.

By “plastic composite” is meant, within the meaning of the invention, a multi-component material comprising at least two immiscible components in which at least one component is a polymer (thermoplastic or thermosetting) and the other component may be a reinforcement such as a fibrous reinforcement.

By “thermoplastic polymer” is meant, within the meaning of the invention, a polymer which is generally solid at room temperature, may be crystalline, semi-crystalline, or amorphous, and which softens during a temperature increase, in particular after passing through its glass transition temperature (Tg), and flows at higher temperatures. Examples of thermoplastics are, for example: low-density polyethylene (LDPE), polyethylene terephthalate (PET), or polyvinyl chloride (PVC).

By “thermosetting polymer” is meant a plastic material that is irreversibly transformed by polymerization into an insoluble polymer network.

By “removable” is meant the ability to be detached, removed, or disassembled easily without having to destroy the means of attachment, either because there is no means of attachment, or because the means of attachment can be easily and quickly disassembled (for example notch, screw, tongue, lug, clips). For example, by removable, is to be understood that the object is not attached by welding or any other means not intended to allow the object to be detached.

By “substantially constant” is meant a value varying by less than 30% with respect to the compared value, preferably by less than 20%, even more preferably by less than 10%.

In the remainder of the description, the same references are used to refer to the same elements.

Existing devices or systems usually have a plurality of sensors (for example pressure sensors) distributed throughout the shoe and/or sole. Such a distribution of sensors leads to a reduction in the robustness of the system. In addition, these devices or systems are usually intended to produce raw data that is then analyzed at an external terminal. Faced with these shortcomings, the inventor developed a system 1 for quantifying or characterizing a user's gait as schematized in FIG. 1.

Also, as a reminder, both feet contain a quarter of all the bones in the human body. In each foot, 26 bones, 33 muscles, 16 joints, and 107 ligaments can be identified. The feet bear the weight of the body in the standing position and allow locomotion, playing a key role in balance, damping, and propulsion. Feet also perform several types of movements. In addition, the feet have almost 7200 nerve endings, so that all diseases and other disorders, especially neurological ones, can be detected directly or indirectly in our feet and, on the other hand, can be detected from the way we walk or move. Generally speaking, any disorder that appears in the human body has an immediate effect on our posture, so that our feet naturally adapt the way we stand on the ground. This is why it is the practice of neurologists to observe their patients' gait before any in-depth examination. This simple observation with the naked eye of the patient's gait already allows the professional to spot or even detect anomalies that would affect the patient's nervous system.

However, existing devices usually only report information relating to biometric parameters that are calculated in delayed mode by external terminals. Faced with these shortcomings, the inventor developed a system 1 for quantifying or characterizing a user's gait, which can be used to detect a disorder or to assist in the detection of a disorder. The advantage of this system is that it is possible to perform real-time characterization or quantification during the individual's conventional or sports walking without the need for controlled conditions or data connections.

Thus, the invention relates to a system of quantifying the gait of a user to obtain a value representative of the evolution of the gait of said user, including an external terminal 20, a pair of soles 10, the soles, constituting said pair of soles, each including an electronic box 100, 101, 102, each electronic box comprising:
an inertial platform configured to generate a set of data on the gait of a user of the pair of soles,
a data processing module configured to transform the generated data set into at least one biomechanical parameter,
a data storage module configured to store the at least one biomechanical parameter,
a first communication means configured so that the electronic box of at least one of the soles is adapted to transmit the at least one biomechanical parameter to the external terminal, and
a power source.

In addition, the external terminal 20 may include a data comparison module configured to compare the at least one biomechanical parameter to reference biomechanical parameters and to calculate a value representative of the evolution of the gait of the user.

In particular, the invention relates to a system of quantifying the gait of a user to obtain a value representative of the evolution of the gait of said user, including a pair of soles, the soles, constituting said pair of soles, each including an electronic box, each electronic box comprising:
an inertial platform configured to generate a set of data on the gait of a user of the pair of soles,
a data processing module configured to transform the generated data set into at least one biomechanical parameter,
a data storage module configured to store the at least one biomechanical parameter,
a data comparison module configured to compare the at least one biomechanical parameter to reference biomechanical parameters and to calculate a value representative of the evolution of the gait of the user,
a first communication means configured so that the electronic box of at least one of the soles is adapted to transmit the at least one biomechanical parameter and/or the value representative of the evolution of the gait of the user to the external terminal, and
a power source.

The system 1 according to the invention includes a pair 10 of soles and an external terminal 20.

The soles 11, 12 that can be used within the context of the system 1 according to the invention may, for example, correspond to outsoles or insoles of shoes. These soles can be removable or permanently integrated into the sole assembly of the shoes. Preferably, the soles are insoles and therefore the electronic boxes are integrated directly into the shoe.

In addition to transverse symmetry, the first shoe sole 11 and a second shoe sole 12 are substantially similar, and therefore only one sole will be described in the present description. The characteristics presented are therefore shared by the first shoe sole and the second sole.

A sole according to the invention may correspond to a multilayer product including an upper layer intended to be in contact, directly or indirectly, with an individual's foot. For example, the sole 11 may correspond to a multilayer product laminated or comprising one or more layers embedded in a polymer such as a thermoplastic (for example polyurethane, ether-ester block copolymer, ether-amide block copolymer, styrene block copolymers). The different layers can be welded together.

The sole, preferably an insole, may have a front portion adapted to be engaged by a forefoot of a user's foot, a middle portion adapted to be engaged by a central part of a user's foot, a rear portion adapted to be engaged by a hindfoot of a user's foot.

For example, the length of the sole is at least twice its width. The thickness of the sole is, for example, at least ten times smaller than its length. For example, the sole may thus be less than one centimeter thick, preferably less than 0.75 centimeters thick, for example about 0.5 centimeters thick.

The sole can advantageously be substantially flat. In addition, it can have a thickness substantially constant over its entire surface. This is particularly advantageous for use in a podiatry context where the insole is then used in addition to a conventional sole, and then an orthopedic insole.

Furthermore, in this context, the system according to the invention may include inserts associated with the soles.

Advantageously, the first shoe sole and the second shoe sole are removable soles.

Alternatively, a first shoe insole can also be permanently integrated into a left shoe, and a second shoe insole can be permanently integrated into the right shoe, for example when making said left shoe and right shoe, as part of the sole assembly of said shoes, for example.

The soles 11, 12 constituting said pair 10 of soles, each include an electronic box 100, 101, 102. As shown in FIG. 1, the electronic box 101, 102 is preferably positioned at a midsole portion. Advantageously, the soles 11, 12 do not include a sensor outside the electronic box. Preferably, the soles 11, 12 do not include a force sensor or pressure sensor.

An electronic box 100 according to the invention is detailed in FIG. 2. Weighing only a few grams and being small in size, this electronic box 100 fits into any insole and/or outsole in a space-saving manner. This low volume limits possible user comfort problems and has the advantage of making it cheaper and simpler to integrate this technology into the sole during the industrial process.

The choice of the material of this electronic box was made in order to ensure its solidity as well as the possibility of inserting it into a sole. Indeed, it should be possible to manufacture a product that can, on the one hand, withstand the weight of a person and, on the other hand, be easily inserted into a sole or shoe. Combining miniaturization and resistance of the box was a real challenge: many prototypes had to be made before determining the material that would allow such a box to be inserted into a sole without altering the comfort thereof.

Advantageously, each electronic box 100 includes an outer casing 103, said outer casing consisting essentially of a material of the plastic composite type selected from: a thermoplastic composite material or a thermosetting composite material.

The plastic composite material preferably has a fibrous reinforcement. The fibrous reinforcement generally refers to several fibers, unidirectional rovings, or a continuous filament mat, fabrics, felts, or non wovens, which may be in the form of strips, webs, braids, wicks, or pieces. Preferably, the fibrous reinforcement of the present invention includes plant fibers, mineral fibers, synthetic polymer fibers, glass fibers, basalt fibers, and carbon fibers, alone or in a mixture. More preferably, the fibrous reinforcement of the present invention includes carbon fibers or glass fibers. More preferably, the fibrous reinforcement of the present invention

The choice of a plastic composite material allows to combine lightness, efficient signal transmission and, above all, solidity.

Thus, each electronic box is preferably light and weighs less than 10 grams, preferably less than 8 grams, and more preferably less than 6 grams. In addition, it may have a thickness of less than 5 mm, preferably less than 4 mm, and more preferably less than 3 mm. This allows it to be easily integrated into a shoe/sole without altering user comfort in the shoe. Finally, each electronic box has less than 5 cm² of surface area on its largest face, preferably less than 4 cm², and more preferably less than 3 cm².

Preferably, the outer casing 103 of the electronic box 100 has an upper part 103a and a lower part 103b which are welded. Such a weld, for example an ultrasonic weld, increases water resistance of the electronic box. Alternatively, the upper part 103a and the lower part 103b can be separated by a polymer seal and held together by removable fastening means. Thus, each electronic box can include an outer casing formed in two parts and a seal positioned between two parts of the outer casing.

Each electronic box advantageously integrates support pillars or pads 104, preferably one pad/cm² to withstand the pressures and impact forces of the movements of the foot. Preferably with a rounded shape to increase its mechanical resistance, it must be assembled in such a way as to maintain a perfect seal and make the interior containing the electronic board and the power source protected from humidity and dust.

Thus, preferably, the electronic box 100 according to the invention includes at least two support pads 104, more preferably at least three support pads 104, and even more preferably at least four support pads 104.

This allows to further strengthen the solidity of the electronic box, and in particular its resistance to pressure. It was also concluded from the tests carried out that support pads were particularly important. Inserting such pads allows the box to better withstand the weight of a person.

Advantageously, the electronic box 100 includes an electronic board with at least one opening 105 allowing the passage of at least one support pad 104, preferably at least two openings 105.

In addition, in order to further increase the robustness of the system, each electronic box includes a shock-absorbing material such as polymer foam (for example polyurethane, polyether). According to one embodiment, the shock-absorbing material has a density between 20 kg/m³ and 50 kg/m³. Such a protective foam layer also allows to insulate the board from vibrations and humidity.

According to one embodiment of the invention, the electronic board is inserted in a compartment of the box specially designed to receive it.

According to another embodiment, the electronic box 100 is formed by the encapsulation of its components. For example, the encapsulation can take the form of an encapsulating coating or a resin (for example silicone, epoxy, polyurethane). The encapsulation of all components (for example inertial platform, processing module . . . ) offers good insulation and thus combines good electrical properties with excellent mechanical protection.

In addition, the electronic box according to the invention includes an inertial platform 110, 111, 112 configured to generate a set of data on the gait of a user of the pair 10 of soles.

In particular, when a user walks, the inertial platform 110 acquires signals representative of a movement parameter (acceleration and/or speed, for example angular velocity) of the foot along the X, Y, Z axes. In addition, this data can then be processed to generate at least one acceleration signal. The inertial platform consists for example of at least one accelerometer and one gyroscope. Preferably, it includes several accelerometers and gyroscopes.

The electronic box may also include one or more magnetometers in order to acquire three additional raw signals corresponding to the values of magnetic fields in three dimensions.

Each electronic box can further include other sensors, including an inclinometer, a barometer, a temperature sensor, and an altimeter for increased accuracy.

It has been observed that too low a sampling frequency results in low reliability, while too high a sampling frequency results in high energy consumption. Thus, preferably, the output signals are sampled at a frequency of at least 25 Hz. The output signals can also be sampled at a frequency of at least 100 Hz, for example at least 200 Hz or 300 Hz. For even greater sensitivity, the output signals can also be sampled at a frequency of at least 400 Hz. However, as mentioned, too high a sampling frequency results in high energy consumption. Thus, the output signals are preferably sampled at a frequency of at most 500 Hz, more preferably sampled at a frequency of at most 250 Hz, and even more preferably sampled at a frequency of at most 150 Hz. For example, the output signals are sampled at a frequency of at least 25 Hz and the output signals are sampled at a frequency of at most 150 Hz. More preferably, the output signals are sampled at a frequency between 30 Hz and 120 Hz, and even more preferably between 50 Hz and 100 Hz. The choice of frequency is made in order to optimize the ratio between energy consumption and reliability of the information acquired.

Preferably, the inertial platform is capable of generating a data set including, for example:
a foot acceleration signal along the X, Y, and/or Z axis,
a foot angular velocity signal around the X, Y, and/or Z axis, and
a magnetic field signal on the X, Y, and/or Z axis.

These six or nine signals can be calibrated or recalibrated, in particular in a fixed reference mark in relation to the ground.

In addition, the electronic box according to the invention includes a data processing module 120, 121, 122 configured to transform the generated data set.

This processing module can be used to pre-process the data set generated by the inertial platform in order to facilitate further processing. Indeed, in the context of the system according to the invention, the generation of the biomechanical parameters of the gait can be carried out by a processing module carried by an external terminal 20.

In addition, the processing module of the box can be used to generate biomechanical gait parameters. Preferably, the data processing module 120 is capable of transforming the data set into at least one biomechanical gait parameter, said biomechanical gait parameter preferably being selected from: posture, pronation, supination, impact force, impact zone, step length, contact time, flight time, limping, propulsion force, balance, and several other parameters relating to the user and describing his/her gait, postures, and movements. Alternatively, or in addition, the processing module of the external terminal 20 can advantageously be configured to perform these actions.

In addition, the transformation by the data processing module may advantageously comprise the segmentation of the data into a plurality of phases. Preferably, the data processing module is capable of segmenting a step into at least four phases such as: the impact phase (corresponds to the precise moment the foot contacts the ground), the support phase (takes place from the impact phase until the heel is detached from the ground), the propulsion phase (begins when the heel has left the ground and ends when the first toe has left the ground), and the flight phase (begins when the first toe has left the ground and ends when the heel touches the ground).

More particularly, cutting or segmenting the step can help identify the main support areas of the user. Thus, the system can be used to measure the shape of the step during walking or any other activity of the user in order to determine possible malformations of the user's feet and postures.

Thus, preferably, the data processing module 120 is adapted to calculate, from the signals generated by the inertial platform, precise biomechanical parameters, representative of the user's gait. As will be detailed later, the calculation of these biomechanical parameters at the sole allows to propose a truly efficient on-board system with much greater autonomy than conventional systems that perform all calculations on external terminals. Moreover, monitoring the evolution of these biomechanical parameters allows the rapid identification of the appearance of a mobility disorder.

Preferably, the data processing module is adapted to, preferably configured to, calculate the values of at least one, for example at least two, of the following biomechanical parameters:
the stability of the foot during the flight phase,
the roll-forward of the step (for example how much time on the heel, support, or propulsion, respectively; or identification of the different phases, time spent on the taligrade, plantigrade, and digitigrade phase, and angles of pronation or supination during each of these phases),
the length of the step (for example corresponds to the progression distance forward of the swinging foot in relation to the other),
the width of the step (for example corresponds to the distance between the innermost parts of two successive footprints during walking),
the angle of the step (for example corresponds to the angle (for example in degrees) open forward formed between the axis of progression and the axis of the foot (heel—second metatarsal)),
the length of the stride (for example corresponds to the progression distance forward of the swinging foot, it usually corresponds to two step lengths for a valid walk),
the width of the stride (for example corresponds to the distance between the innermost parts of two successive footprints of a same foot during walking),
The trajectory of the step (for example characterization of the foot movement during the swing phase such as height, width), and/or
The pace: corresponds to the number of steps per minute.

More preferably, the data processing module is adapted to calculate the values of at least one, for example at least two, of the following biomechanical parameters: stability of the foot during the flight phase, step roll-forward, step length, step width, step angle, stride length, and/or stride width.

Even more preferably, the data processing module is adapted to calculate the values of at least one, for example at least two, of the following biomechanical parameters: step roll-forward, stride length, stride width, and step angle.

This constitutes a list of different biomechanical parameters and the invention is not limited to the calculation of these particular parameters. Indeed, from the data generated by the inertial platforms, the invention allows to calculate a plurality of different biomechanical parameters, the list of which is limited only by their usefulness to the user.

For example, the data processing module may be adapted to, preferably configured to, calculate a propulsion orientation value. This biomechanical parameter corresponds more particularly to the angle of a foot, for example in relation to the ground, during the propulsion phase. Similarly, the data processing module may be adapted to, preferably configured to, calculate a value for many other biomechanical parameters.

In addition, in the context of the present invention, the data processing module can be adapted to, preferably configured to, calculate a value of a so-called synchronized biomechanical parameter. Within the meaning of the invention, a so-called synchronized biomechanical parameter is a biomechanical parameter, the calculation of which requires data from the two electronic boxes. Thus, in this context, a second box can be configured to transmit to the first electronic box data generated by its inertial platform or one or more biomechanical parameters, and the first electronic box is then configured to generate a biomechanical parameter value so-called synchronized from the data generated by the inertial platform of the second electronic box or one or more biomechanical parameters calculated by the second electronic box. This embodiment is particularly advantageous because it allows access to fine characterizations of the gait while using an energy-efficient on-board sensor system.

Furthermore, in the context of the present invention, the data processing module can be adapted to, preferably configured to, calculate a combinatorial pattern of biomechanical parameters. Within the meaning of the invention, a combinatorial pattern of biomechanical parameters corresponds to a combination of biomechanical parameters (i.e. values) or to a time-dependent behavioral combination of biomechanical parameters. Such a combinatorial pattern of biomechanical parameters can be advantageously associated with a physiological state of the user. Thus, in this context, the first box and/or the second box can be configured to compare a combinatorial pattern of biomechanical parameters to a combinatorial pattern of reference biomechanical parameters. Alternatively, they can be configured to transmit to the external terminal a combinatorial pattern of biomechanical parameters. This embodiment is particularly advantageous because it allows new patterns that can be correlated to predetermined physiological states or pathological conditions to be generated and thus access, from a characterization of the gait, to risk data for the user. Alternatively, the calculation of a combinatorial pattern of biomechanical parameters, and then its comparison can be made by the comparison module carried by the electronic box or the external terminal.

A combinatorial pattern of biomechanical parameters may, for example, include a combination of a pace value, a stride length value, and a walking speed. Such a combinatorial pattern of biomechanical parameters allows from the individual values of each of these three parameters to determine a walking disorder that can be caused, for example, by an aggravation of a Parkinsonian step.

For example, the system 1 according to the invention can be configured to record from the first uses the average step length of the user and monitor the evolution of this length. Depending on the age of the user, this length may increase or decrease, but this evolution will happen gradually and noticeably. However, if the invention should detect a sudden change in this step length, it will interpret it as an anomaly likely to reveal a physical or other disorder in the user.

From then on, the user will be alerted by the system according to the invention, even though the detected disorder has remained imperceptible to him/her. This will also be the case when the user's gait becomes shaky, or when a slight limp appears in his/her walk . . . .

From then on, the user will be able, in advance, to consult any health professional of his/her choice to carry out medical examinations for checking whether or not these evolutions correspond to the emergence of a pathology or malformation.

In addition, the data processing module according to the invention is adapted to calculate an asymmetry between the biomechanical parameters of a right leg with respect to the biomechanical parameters of a left leg.

In addition, the data processing module according to the invention is adapted to calculate the variability of biomechanical parameters associated with one leg or both legs.

Advantageously, the processing module is adapted to establish a user profile during a first period of use. This first period of use can, for example, last a day, a week, or a month. A first period of use is preferably of sufficient duration to calculate a set of biomechanical parameters stable over time with preferably low variability (for example less than 20%, preferably less than 10%). It usually takes a few days to a few weeks to establish a user profile.

Preferably, the processing module is configured to establish a profile of the user's biomechanical parameters including at least one of the following parameters: step length, impact force, pace (step frequency), and time of flight. Preferably, the user's biomechanical parameter profile should include at least two, more preferably at least three, of the following parameters: step length, impact force, pace, and time of flight.

Thus, the system 1 can also be used by chiropodists or physicians to analyze the user's biomechanical parameter profile in order to propose appropriate solutions. This analysis can also be done with athletes to reduce the risk of injury or improve their performance, or in the context of a professional activity to detect the hardship of a work station. The system 1 according to the invention could then serve as a mobile scanning or analysis tool capable of providing real-time data.

When an electronic box is notable to communicate in real time with the other box and/or with the terminal, it stores the collected information and will transmit it in delayed mode when the exchange is possible again. This delayed transmission of the collected data is made possible using the storage capacity each of the electronic boxes is provided with.

Thus, the electronic box according to the invention includes a data storage module 130, 131, 132, configured to store at least part of the transformed data and/or generated data. More particularly, it is configured to store the biomechanical parameters calculated by the processing module 120 as well as the reference biomechanical parameters used by the comparison module 140. It can also be configured to store values representative of the user's balance evolution. It can also be configured to store the data generated by the inertial platform. Advantageously, the data storage module 130, 131, 132 is configured to store at least part of the transformed data but not to store the generated data. Thus, its capacity is not burdened by generated raw data. The transformed data can correspond to data pre-processed by the processing module or to biomechanical parameters.

In addition, the electronic box may include a data comparison module 140, 141, 142 configured to compare at least one biomechanical parameter to reference biomechanical parameters and it is adapted to calculate a value representative of the evolution of the user's gait. This can allow it to quantify a user's gait and more particularly to identify a gait disorder.

Alternatively, the data comparison module 140, 141, 142 can be carried by the external terminal 20. In this case, it is also configured to compare at least one biomechanical parameter with reference biomechanical parameters, and it is adapted to calculate a value representative of the evolution of the user's gait.

Preferably, the electronic box includes a data comparison module 140, 141, 142 configured to compare at least one biomechanical parameter with reference biomechanical parameters and it is adapted to calculate a value representative of the evolution of the user's gait.

Advantageously, the comparison is made in real time. Thus, the external terminal 20 will detect deficiencies or anomalies that appear in the user's walk or, more generally, in the user's gait. As regards the box, it will not only be able to note the different positions, but also to detect deficiencies or anomalies that appear in the user's walk, or more generally the user's gait. The comparison can be made with different reference data. Thus, for example, the reference data is selected from:
transformed data, or values of biomechanical parameters, previously obtained from the user using the system according to the invention,
transformed data, or values of biomechanical parameters, representative of pathologies, for example obtained from individuals with at least one pathology that may affect gait, and
predetermined threshold values characteristic of certain pathological conditions.
   Prior Transformed Data Obtained from the Individual Using the System According to the Invention:

Here the objective is to detect a continuous evolution in a person.

The data comparison module 140, of the box or of the external terminal 20, is particularly suitable for monitoring over time, preferably continuously, the evolution of the calculated biomechanical parameters of a user. The module can also be configured to make a comparison on a daily, weekly, or monthly basis.

Advantageously, the data comparison module 140, of the box or of the external terminal 20, is adapted to compare the calculated data of biomechanical parameters with the user profile.

Thus, the system according to the invention will, for example, be able to identify or detect a decrease in a user's step length over time. Such a decrease is generally not normal and should alert the user who should then be able to take preventive or corrective action.

Transformed Data Obtained from Individuals with at Least One Pathology that can Affect Gait

Here the objective is to verify if the biomechanical parameters calculated for an individual using the system are not similar to biomechanical parameters associated with walks or gaits considered pathological or pre-pathological or at risk of developing a pathology.

Thus, the data comparison module 140, of the box or of the external terminal 20, according to the invention, is configured to compare the transformed data to reference data (for example reference biomechanical parameter) including biomechanical parameters representative of pathologies or risks of developing pathologies.

For example, the transformed data can be compared to biomechanical parameters indicative of risks or the presence of neurological pathologies such as Parkinson's disease, Huntington's disease, Charcot's disease, neuromuscular pathologies with in particular Duchenne de Boulogne muscular dystrophy, muscular pathologies such as muscular tears or dystrophies, joint pathologies such as sprains, traumatic meniscus injuries or arthritis, or podiatric pathologies such as tendinopathies, scoliosis, or bow legs.

More specifically, the biomechanical reference parameter values can be associated with recognized pathologies, with the gaits then being selected from: Parkinsonian steps, Huntington's chorea, normal pressure hydrocephalus, limping, saluting walk, radicular intermittent claudication, waddling walk, mowing walk, stepping walk, walk with retropulsion, heeling walk, vestibular walk, spasmodic walk, lightheadedness walk, walking ataxia, hesitant walk, painful walk, “small steps” walk, or trembling walk.

The system according to the invention does not allow for a diagnosis. Nevertheless, it allows the generation of an alert aiming to warn a user of a disorder in his/her gait that may need to be further investigated, for example by resorting to a doctor.

For example, in the context of Parkinson's disease, it is now established that certain walking biomechanical parameters are associated with this disease. In a person with Parkinson's disease can be observed a stooped gait with small steps, or the lack of initiation, the phenomenon of festination, and a postural instability.

The system, by means of the various sensors of the inertial platform, is able to detect small steps and calculate the time of flight, the contact time, as well as the length of the step. If the contact time is longer than the time of flight in this case, small steps are characterized. Postural instability will be detected by several parameters such as those related to walking with retropulsion. In this case, a step roll-forward measurement is carried out to determine the value of the peaks, i.e. heel and toe placement, and thus establish thresholds. If the threshold is reached, postural instability can be characterized.

Furthermore, in the context of Duchenne muscular dystrophy, a neuromuscular disease resulting in degeneration of muscle tissue and presenting in particular as symptoms an awkward gait and frequent falls, the system 1 according to the invention may be configured to measure and record the falls and risks of falls to which the user has been exposed.

In particular, the data comparison module, of the box or of the external terminal 20, can be configured to generate a data item selected from:
an efficiency index of a care protocol: corresponds, for example, to a value that can help a hospital practitioner to identify a progression in a patient's gait, which he/she can then use within the framework of his/her own methods in order to evaluate the effectiveness of a treatment;
a data item of the nature of the support: corresponds for example to the way the foot presents itself on the ground: by the heel, the arch of the foot, or the toes;
a data item of the step profile: corresponds for example to the impact force, the time of the impact on the ground, the pace, or a limping problem, that is to say an imbalance between right and left foot;
a data item of the walking technique: corresponds to the roll-forward of the step (support phase and swing phase), which is broken down into heel strike, braking phase, valgus thrust, and propulsion phase;
a data item of the support area: corresponds for example to pronation or supination;
a data item of a corrective solution: corresponds, for example, to a corrective solution such as corrective inserts or a solution such as recommended exercises.

In addition, the electronic box according to the invention includes a first communication means 150, 151, 152 configured so that the electronic box 100 of at least one of the soles is capable of transmitting at least part of the transformed data to an external terminal 20. This data can be transmitted in real time or in delayed mode to an external terminal 20. The external terminal 20 can for example be a remote system such as a tablet, a mobile phone (“smartphone” in Anglo-Saxon terminology), a computer or a server.

Advantageously, each electronic box further includes a second communication means configured so that the electronic box 101 of a first sole is able to communicate with the electronic box 102 of a second sole, and so that at least one data processing module 121, 122 is configured to calculate, preferably jointly, sets of data generated from the two soles 11, 12, and more particularly from the inertial platform, constituting the pair 10 of soles. Indeed, the calculation of certain biomechanical parameters of interest requires the data from both soles.

For example, the first and the second box can calculate provisional values of biomechanical parameters and the second box is advantageously configured to transmit said provisional values of gait parameters to the first box. As regards the first box, it is configured to compare the provisional values of biomechanical parameters of the second box to those of the first box in order to generate a consolidated value of biomechanical parameters.

The first and second communication means are adapted to receive and transmit the data over at least one communication network. Preferably, the communication is operated via a wireless protocol such as WiFi, 3G, 4G, and/or Bluetooth.

In addition, the electronic box according to the invention includes a power source 160, 161, 162. The power source is preferably of the battery type, rechargeable or not. Preferably, the power source is a rechargeable battery. In addition, it can be combined with a system for recharging by movement or with external energy. In particular, the system for recharging with external energy can be a wired recharging system, or an induction recharging system.

In addition, the electronic box according to the invention may include a wired connection means 180, preferably protected by a removable tab. This wired connection can be for example a USB or FireWire port. This wired connection means can be used as mentioned above to recharge the battery but also to exchange data and for example to update the firmware of the electronic board carrying the various components of the electronic box.

Indeed, the various components of the electronic box are preferably arranged on an electronic board 170 (or printed circuit). In addition, the various means and modules of the electronic box 100 are represented separately in FIGS. 1 and 2, but the invention may provide for various types of arrangement such as, for example, a single module combining all the functions described here. Similarly, these means can be divided into several electronic boards or gathered on a single electronic board. In addition, when an action is given to a device, a means, or a module, it is actually performed by a microprocessor in the device or module controlled by instruction codes stored in a memory. Similarly, if an action is given to an application, it is actually performed by a microprocessor in the device, in a memory of which the instruction codes corresponding to the application are stored. When a device or module sends or receives a message, this message is sent or received by a communication interface.

In addition, the system 1 includes an external terminal 20 adapted to receive data such as at least one biomechanical parameter and/or the value representative of the user's gait evolution. In addition, the external terminal 20 may itself include a comparison module or a processing module and a comparison module. Thus, it is based on pre-processed data generated by the electronic boxes to calculate an evolution value of the user's gait.

Thus, the user can access data related to his/her daily physical activities and related to several biomechanical parameters, such as posture, pronation/supination, impact force, step length, contact time, limping, balance, and several other parameters related to the user and describing his/her movements, walk, postures, and motion, and thus follow their evolution.

But above all, it can access comparative data, or values representative of the evolution of the gait, which can be relative, on the one hand, to a comparison over time of the values of biomechanical parameters to monitor their evolution and detect anomalies that may be linked to the emergence of a pathology or malformation and, on the other hand, to compare these data with other parameters recognized as being associated with pathologies and to alert the user in the event of a significant similarity revealing a pathology or malformation and, finally, to enable a health professional to detect a malformation and/or to monitor the effects of a medical treatment prescribed to a patient.

The external terminal 20 is usually a tablet, a mobile phone (“smartphone” in Anglo-Saxon terminology), a computer, or a server. It may be able to transfer this data to a remote server 30. It is then possible, for example, to access this remote server via a web interface. All communications with the remote server can be secured, for example by HTTPS protocols and AES 512 encryption. Thus, this may allow, via a client, access to data by medical staff in charge of monitoring the user.

In addition, particularly in the context of calculating a value representative of the evolution of the gait, the data comparison module 140 and/or the external terminal 20 may, for example, be able to implement algorithms based on supervised or unsupervised learning methods. Thus, advantageously, the system 1 is configured to implement the values of biomechanical parameters in one or more algorithms, preferably pre-calibrated. These algorithms may have been built from different learning models, in particular partitioning, supervised, or unsupervised models. An unsupervised learning algorithm can for example be selected from an unsupervised Gaussian mixture model, a hierarchical bottom-up classification (Hierarchical clustering Agglomerative in Anglo-Saxon terminology), a hierarchical top-down classification (Hierarchical clustering divisive in Anglo-Saxon terminology). Alternatively, the algorithm is based on a supervised statistical learning model configured to minimize a risk of the ordering rule and thus allowing more efficient prediction rules to be obtained. In this case, the calculation, determination, and estimation steps can be based on a model, trained on a data set, and configured to predict a label (for example similar or dissimilar to the registered gait). For example, for calibration purposes, it is possible to use a data set that is representative of a situation with a known label, such as biomechanical parameters characteristic of Parkinson's disease. The data set can also comprise multiple labels. The algorithm can be derived from the use of a supervised statistical learning model selected for example from kernel methods (for example separators Wide Margin—Support Vector Machines SVM, Kernel Ridge Regression), set methods (for example decision trees), hierarchical partitioning, k-mean partitioning, decision trees, logical regression, or neural networks.

The comparison module 140 and/or the external terminal 20 may also be able to compare, preferably in real time, the data of biomechanical parameters to predetermined critical thresholds of biomechanical parameters or of the value representative of the evolution of the gait and to generate an alert according to the result of the comparison. This allows the system to identify potential risks and for example gaits that deviate from the norm.

Thus, the system 1 according to the invention will be able to analyze the values generated and to refine the analysis so as to interpret the evolution of the values of the parameters over time in order to identify or not an anomaly in the gait possibly indicative of a risk or a pathology or a potential malformation. As soon as the system 1 finds a similarity between the user's data and monitoring parameters characteristic of one or more of the identified pathologies, it will be able to alert the user by a message on the remote terminal and invite him/her to contact a health professional for further examination.

In addition, the system 1 according to the invention can be used in the context of a prognostic procedure so as to monitor, for example, the effectiveness of treatments or care protocols prescribed to patients in the context of specific pathologies.

For example, at present, when a health professional prescribes a treatment to his/her patient, he/she must wait for the next two or three appointments with that patient in order to be able to evaluate the effectiveness of the prescribed treatment based on biological examinations and/or sensations reported by said patient. This method of evaluation, in relation to certain neurological pathologies, may prove to be random.

Multiple sclerosis is a disease characterized by repeated, regressive (at least at the beginning of the disease) neurological accidents affecting variable functions (vision, motor skills, sensitivity, etc.), the flare-ups of which are scattered in time and space. As there is currently no cure for this disease, the prescribed treatments are aimed at improving function after a flare-up or postponing new attacks. In this hypothesis, the health professional is relatively powerless to follow up with his/her patient. He/she also has no way to ensure that the treatment he/she has prescribed has a beneficial effect on this patient. The invention overcomes this difficulty.

In another example, associated with a myopathy, in the event that a health professional proposes a particular treatment to a patient suffering from this pathology, he/she will be able, thanks to the system 1 according to the invention, to take note of the evolution of his/her patient's gait and evaluate the effectiveness of the prescribed treatment. If a reduction in collisions and falls is observed in this patient, the professional may conclude that the patient's condition has improved as a result of the effectiveness of the treatment. Otherwise, if the frequency of collisions and falls remains the same or increases, the professional will be able to observe the deterioration of the patient's condition and conclude that the treatment is ineffective.

In addition, the external terminal 20 or the remote server 30 may include:
A calculation module adapted to study the biomechanical data collected on a daily basis and analyzing the evolution of this data over time. For example, the calculation module can monitor the evolution of this data over time, which will be linked to the user's age and his/her daily physical activities,
an alert module adapted to alert the user, and possibly medical staff, of an abnormal evolution of one or more biomechanical parameters that may correspond to the appearance or an increased risk of the appearance of one or more pathologies,
a communication module adapted to directly contact and inform a health professional or any other person chosen and previously indicated by the user in the application and/or adapted to regularly provide the user with information related to his/her daily activity and the evolution of the biomechanical parameters over time,
a corrective module adapted to propose, for example via the external terminal, solutions to rectify or prevent malformation by proposing physical exercises or corrective inserts to be integrated under the foot,
a prognostic module adapted to measure the effect, and possibly the effectiveness, of a neurological, medical, or other treatment protocol by following the evolution of all or part of the walking parameters via, for example, the value representative of the evolution of the user's gait and adapted to communicate to the user or directly to his/her doctor or any other predetermined person all or part of the observed evolutions of the data collected from said user's gait.

According to another respect, the invention relates to a method of quantifying the gait of a user. Preferably, the quantification method is implemented using a quantification system according to the invention.

The method according to the invention can implement a quantification system including a pair 10 of soles and an external terminal 20.

In addition, the soles 11, 12, constituting said pair 10 of soles, may each include an electronic box 100, 101, 102, each electronic box 100, 101, 102 comprising an inertial platform 110, 111, 112, a data processing module 120, 121, 122, a data storage module 130, 131, 132, a first communication means 150, 151, 152, and a power source 160, 161, 162.

In addition, each electronic box 100, 101, 102 can comprise a data comparison module 140, 141, 142.

The quantification method according to the invention includes the following steps:
Generating 210, by the inertial platform 110, 111, 112, a set of data on the gait of a user of the pair 10 of soles,
Transforming 220, by the data processing module 120, 121, 122, the generated data set into at least one biomechanical parameter,
Storing 230, by the data storage module 130, 131, 132, at least one biomechanical parameter,
Comparing 240 the at least one biomechanical parameter to reference biomechanical parameters,
Calculating 250 a value representative of the evolution of the gait of the user,
Transmitting 260, by a first communication means 150, 151, 152 of at least one of the soles, the at least one biomechanical parameter and/or the value representative of the evolution of the gait of the individual to the external terminal 20.

In particular the invention relates to a method of quantifying the gait of a user implementing a quantification system including a pair of soles and an external terminal, the soles constituting said pair of soles, each including an electronic box, each electronic box comprising an inertial platform, a data processing module, a data storage module, a data comparison module, a first communication means, and a power source, said method including the following steps:
Generating 210, by the inertial platform 110, 111, 112, a set of data on the gait of a user of the pair 10 of soles,
Transforming 220, by the data processing module 120, 121, 122, the generated data set into at least one biomechanical parameter,
Storing 230, by the data storage module 130, 131, 132, at least one biomechanical parameter,
Comparing 240, by the data comparison module 140, 141, 142, the at least one biomechanical parameter to reference biomechanical parameters,
Calculating 250, by the data comparison module 140, 141, 142, a value representative of the evolution of the gait of the user,
Transmitting 260, by a first communication means 150, 151, 152 of at least one of the soles, the at least one biomechanical parameter and/or the value representative of the evolution of the gait of the individual to the external terminal 20.

The method according to the invention further comprises a step of communicating between the first and second box, comprising transmitting, by a communication module, transformed data from the second box to the first box. This step of communicating between the first and second box can be carried out according to a predefined time frequency, for example the transmission can be carried out every second, or every two seconds, or for any other predefined time frequency. This step allows to gather all the data on a single box, preferably on the first box. In addition, this communication step may include transmitting provisional values of biomechanical parameters from a second box to a first box, or vice versa.

The method comprises a step of selecting, by the first box, a movement category representative of the raw data generated by the first box and those of the second box. The method also comprises a step of processing provisional values of biomechanical parameters of the second box and the first box in order to select values of biomechanical parameters to be retained.

In addition, the method according to the invention also includes a step of comparing provisional values of biomechanical parameters of the second box to those of the first box so as to generate a consolidated value of biomechanical parameters for the period of time. Preferably, the method may then also include a step of transmitting the biomechanical parameter value to an external terminal. This transmission is preferably made on an ad hoc basis. The transmission frequency can therefore be greater than 100 ms, preferably greater than 1 second. For example, the transmission is every 10 seconds. The method comprises a step of storing the biomechanical parameter value by the first box. Advantageously, in contrast to the raw and/or pre-processed data that is saved for a short time (for example less than 5 minutes), for example on a cache, the new advanced gait parameter value is stored for a longer time, for example on a memory.

In addition, the method according to the invention may include a step of identifying areas of support and the roll-forward of the step when a user moves (walking or running). Such a step allows to quantify the level of stress on each of the user's feet and possibly prevent deterioration of the user's gait.

The method according to the invention may also include a step of determining the roll-forward of the step when a user moves. More particularly, it may include a step of defining the user's step profile.

In particular in this context, the invention may make it possible to propose solutions for correcting the user's gait in order to prevent the appearance of malformations. Thus, the method according to the invention may include a selection of exercises which may improve said roll-forward of the step and possibly reduce physical fatigue or the risk of injury to the user, a selection of inserts to be put in place, or a selection of other footwear products which are more suitable.

In addition, the method according to the invention may also include a step of evaluating the evolution of a disease. Thus, a practitioner will be able to validate and/or adapt a treatment.

An advantage of the present invention is that it specifically enables chiropodists to characterize the walk or gait of a patient, in order to produce adapted inserts for his/her benefit.

In this context, according to another aspect, the invention relates to a method of designing orthopedic soles including the following steps:
a step of implementing a method of quantifying the gait according to the invention, and
a step of defining the structure of orthopedic soles depending on the monitoring data obtained during the implementation step.

In this context, according to another aspect, the invention relates to a method of manufacturing orthopedic soles including the following steps:
a step of implementing a method of quantifying the gait according to the invention,
a step of defining the structure of orthopedic soles depending on the monitoring data obtained during the implementation step, and
a step of manufacturing the orthopedic soles defined in the previous step.

In the context of methods of designing and manufacturing orthopedic soles, the step of defining the structure of orthopedic soles may include running a program for three-dimensionally modeling the shape of the sole based on the monitoring data obtained during the quantification method. Furthermore, this step may further include defining the shape of the sole and/or the density of different areas of the sole so as to take into account data generated by the system according to the invention, such as previously generated support area data. This three-dimensional modeling can also be corrected manually using manual measurements in order to obtain a model best suited to the future user's gait. This 3D model is defined by data (spatial coordinates) that can be used for example by a machine tool, for example a numerical cutting machine-tool, configured to manufacture at least part of the sole (for example vamp, tongue, quarters). These cut parts can then be assembled to form an orthopedic sole or possibly a shoe including said orthopedic sole.

As detailed above, the present invention allows to monitor the evolution of biomechanical data over time in order to be able to detect any abnormal evolution by comparison to the values of these data collected at the time of the first use and during subsequent uses. Furthermore, the present invention allows to analyze the biomechanical data and to compare them to determined monitoring parameters, in order to detect in the user a risk of developing a pathology or malformation (in particular in children). It can also measure the user's fatigue and risk of joint or muscle injuries: if he/she runs with force on the toes, there is a high risk of muscle injuries; conversely, if he/she runs with strong pressure on the heel, the injuries incurred will be joint-related.

Advantageously, the invention intervenes here as an upstream detection system, capable of detecting the slightest anomaly in the user's gait or posture, and revealing the appearance of a specific disorder. Thus, without being a method for making a diagnosis, the present invention may enable a health professional, in the context of determined pathologies (neurological diseases, in particular), to detect an increased risk of developing the pathology, to monitor the effectiveness of a treatment or care protocol prescribed to a patient and also to monitor the rehabilitation of a patient (for example a football player). The health professional will then have a set of biomechanical information about the patient, the analysis and interpretation of which will lead to a more relevant understanding of the patient's disorders. The professional will be able to refine his/her diagnosis and adapt the care protocol according to the information transmitted to him/her from the patient's biomechanical data.

Because of all the advantages of the present invention, it is also possible to monitor in real time the evolution of the user's gait in order to prevent any deterioration in his/her health condition as quickly as possible. In particular, this is possible through a less complex, more robust, and more autonomous system than those used until now.

All these benefits therefore contribute to improving functioning and reducing pathological risks.

1. A system of characterizing a gait of a user to obtain a value representative of an evolution of the gait of said user, including a pair of soles and an external terminal, each of said soles including an electronic box, each said electronic box comprising:
an inertial platform configured to generate a set of data on the gait of a user of the pair of soles,
a data processing module configured to transform the generated data set into at least one biomechanical parameter,
a data storage module configured to store the at least one biomechanical parameter, and
a power source;
said system including a data comparison module configured to compare the at least one biomechanical parameter to reference biomechanical parameters and to calculate a value representative of the evolution of the gait of the user;
said data comparison module being carried by the electronic boxes or the external terminal; and
each said electronic box further comprising a first communication means configured so that the electronic box of at least one of the soles is adapted to transmit the at least one biomechanical parameter and/or the value representative of the evolution of the gait of the user to the external terminal.

2. The system according to claim 1, wherein each said electronic box further includes a second communication means configured so that the electronic box of a first sole of said pair of soles is adapted to communicate with the electronic box of a second sole of said pair of soles, and at least one of the data processing modules is configured to transform sets of data generated from the two soles constituting the pair of soles into the at least one biomechanical parameter.

3. The system according to claim 1, wherein the data comparison module is configured to perform a monitoring over time of the evolution of the calculated value representative of the evolution of the gait of the user.

4. (canceled)

5. The system according to claim 1, wherein the transformation by the data processing module comprises segmenting the data into a plurality of step phases.

6. The system according to claim 1, wherein the reference biomechanical parameters to which the at least one biomechanical parameter is compared are biomechanical parameters previously generated by the same user of the system.

7. The system according to claim 1, wherein the reference biomechanical parameters to which the at least one biomechanical parameter is compared are predetermined biomechanical parameters, associated with one or more mobility disorders.

8. The system according to claim 1, wherein the data processing module is adapted to calculate the values of at least two of the following biomechanical parameters: stability of the user's foot during a flight phase, step roll-forward, step length, step width, step angle, stride length, and/or stride width.

9. The system according to claim 1, wherein a second one of the electronic boxes is configured to transmit, to a first one of the electronic boxes, data generated by its inertial platform or one or more biomechanical parameters, and the first said electronic box is then configured to generate a biomechanical parameter value so-called synchronized in particular from:
the least one biomechanical parameter obtained by the first electronic box, and
data generated by the inertial platform of the second electronic box or one or more biomechanical parameters calculated by the second electronic box.

10. The system according to claim 1, wherein the data processing module or the comparison module is further configured to calculate a combinatorial pattern of biomechanical parameters.

11. The system according to claim 1, wherein the data processing module is adapted to calculate an asymmetry between the biomechanical parameters of a right leg relative to the biomechanical parameters of a left leg.

12. The system according to claim 1, wherein the data processing module is adapted to calculate a variability of the biomechanical parameters associated with one leg or both legs.

13. The system according to claim 1, wherein the data processing module is adapted to establish a profile of biomechanical parameters of the user including at least one of the following parameters: step length, step angle, impact force, pace, and time of flight.

14. The system according to claim 1, wherein the data comparison module is adapted to generate a data item from: an efficiency index of a care protocol, a data item of the nature of a support, a data item of a step profile, a data item of a walking technique, a data item of a support area, and a data item of a corrective solution.

15. The system according to claim 1, wherein the data comparison module is configured to generate an efficiency index of a care protocol.

16. The system according to claim 1, wherein the data comparison module is configured to generate a data item of a corrective solution.

17. The system according to claim 1, wherein the data storage module is configured to store at least part of the at least one biomechanical parameter transformed from the generated data but not to store the generated data.

18. The system according to claim 1, wherein the data comparison module is configured to detect a decrease in a user's step length over time.

19. The system according to claim 1, wherein the biomechanical reference parameter values are associated with recognized pathologies having characteristic gaits selected from the group consisting of: Parkinsonian steps, Huntington's chorea, normal pressure hydrocephalus, limping, saluting walk, radicular intermittent claudication, waddling walk, mowing walk, stepping walk, walk with retropulsion, heeling walk, vestibular walk, spasmodic walk, lightheadedness walk, walking ataxia, hesitant walk, painful walk, “small steps” walk, and trembling walk.

20. A method of characterizing a gait of a user implementing a quantification system including a pair of soles and an external terminal, each of said soles including an electronic box, each said electronic box comprising an inertial platform, a data processing module, a data storage module, a first communication means, and a power source, said system including a data comparison module carried by the electronic boxes or the external terminal, said method including the following steps:
Generating, by the inertial platform of at least one of the soles, a set of data on the gait of a user of the pair of soles,
Transforming, by the data processing module of at least one of the soles, the generated data set into at least one biomechanical parameter,
Storing, by the data storage module of at least one of the soles, the at least one biomechanical parameter,
Comparing, by the data comparison module, the at least one biomechanical parameter to reference biomechanical parameters,
Calculating, by the data comparison module, a value representative of an evolution of the gait of the user,
Transmitting, by a first communication means of at least one of the soles, the at least one biomechanical parameter and/or the value representative of the evolution of the gait of the user to the external terminal.

21. A method of designing orthopedic soles including the following steps:
implementing the method for characterizing the gait of a user according to claim 20, and
defining a shape and ergonomics of orthopedic soles depending on monitoring data obtained during the implementation stage.
